(19) United States
(12) Patent Application Publication
(10) Pub. No.: US 2015/0335262 A1
(43) Pub. Date: Nov. 26, 2015

(54) SYSTEM, METHOD AND COMPUTER-ACCESSIBLE MEDIUM FOR THE PROBABILISTIC DETERMINATION OF NORMAL PRESSURE HYDROCEPHALUS

(71) Applicant: New York University, New York, NY (US)

(72) Inventors: Ajax George, New York, NY (US); Henry Rusinek, Great Neck, NY (US)

(73) Assignee: New York University

(21) Appl. No.: 14/719,960

(22) Filed: May 22, 2015

Related U.S. Application Data
(60) Provisional application No. 62/001,931, filed on May 22, 2014.

(51) Int. Cl. A61B 5/055; A61B 5/00

(52) U.S. Cl. CPC A61B 5/055; A61B 5/7275

(57) ABSTRACT

Exemplary systems, methods, and computer-accessible mediums can be provided for determining a probability or a presence of a disease(s), which can include, for example, receiving information related to an image(s) of a brain of a patient(s), and determining the probability or the presence of the disease(s) in the patient(s) based on ventricular volume and gray matter of the brain. The disease can be normal pressure hydrocephalus or Alzheimer disease. The determining procedure can be based on the probability, which can be based on a prediction model(s). The prediction model can be a multinomial regression model. The image can be a magnetic resonance image of the brain of the patient(s

### CROSS-REFERENCE TO RELATED APPLICATION(S)

This application relates to and claims priority from U.S. patent application Ser. No. 62/001,931, filed on May 22, 2014, the entire disclosure of which is incorporated herein by reference.

### FIELD OF THE DISCLOSURE

The present disclosure relates generally to the diagnosis of a disease, and more specifically, to exemplary embodiments of exemplary system, method, and computer-accessible medium for the probabilistic determination of normal pressure hydrocephalus.

### BACKGROUND INFORMATION

The term—normal pressure hydrocephalus (“NPH”)—was proposed to define a disorder in patients with cognitive impairment, gait dysfunction and urinary incontinence who showed enlarged ventricles by pneumoencephalography despite normal intracranial pressure. (See, e.g., References 1 and 2). Patients diagnosed to have NPH, responded to cerebral spinal fluid (“CSF”) drainage by spinal tap and showed symptomatic improvement following shunt surgery. (See, e.g., Reference 2). Since 1965, NPH has been recognized as a treatable cause of motor deficits, cognitive impairment and urinary incontinence. (See, e.g., Reference 3). Approximately 50% of patients who receive ventricular shunting show sustained clinical improvement over a five year period. (See, e.g., Reference 4). Several other studies have shown a rate of improvement ranging from about 24% to about 80%. A recent meta-analysis demonstrated a mean improvement rate of about 59% in idiopathic NPH following a shunt procedure. (See, e.g., References 5-9).

NPH can develop as a consequence of prior central nervous system pathology such as subarachnoid hemorrhage, meningitis or traumatic brain injury. In this setting the diagnosis can be relatively straightforward, and patients usually respond favorably to shunt placement. NPH in its idiopathic form, however, can be far more common, and can often be a cause of incapacitating gait instability and progressive dementia in older adults. (See e.g., Reference 10).

Identifying NPH in magnetic resonance images or imaging (“MRI”) of elderly patients in day-to-day practice, and differentiating NPH from cerebral atrophy or other neurodegenerative diseases, in particular Alzheimer disease (“AD”), remains a challenge. This can be due to the overlapping clinical and imaging features, the similar age group they affect, and the variability among radiologists in the interpretation of MRI scans of elderly patients. (See e.g., Reference 11). Identifying NPH patients that can benefit from shunt surgery can be particularly difficult.

Unfortunately, ventricular enlargement, a hallmark of NPH, can also be a characteristic of other neurodegenerative diseases as well as a normal sign of aging. Although various imaging signs such as: (i) rounding of the frontal horns, (ii) stretching and bowing of the corpus callosum, (iii) compression of the interpeduncular fossa, (iv) upward displacement of the superior parietal lobule, and (v) expansion of the Sylvian fissures (“SF”) and compressed as well as asymmetrically enlarged cortical sulci (“CS”) that can paradoxically collapse after shunt (see, e.g., Reference 12), can help distinguish NPH from other causes of cerebral atrophy, these signs can be subjective, and may not be universally present in NPH. Additionally, the widely referenced Evans' index (“EI”), the ratio of the transverse diameter of the anterior horns of the lateral ventricles to the greatest internal diameter of the skull, has been shown to have limited reliability. (See, e.g., Reference 13).

Gray matter (“GM”) volume loss measured with high-resolution MRI can be another marker for neurodegeneration. It has been demonstrated that global GM loss in AD patients can be used to quantify previously reported subjective observation of reduced gray-white matter discrimination. (See, e.g., References 14 and 15).

The differentiation between NPH, cerebral atrophy and normal aging also poses a challenge in day-to-day MRI and computed tomography (“CT”) interpretation. Evidence suggests that a large number of patients incapacitated by NPH can be misdiagnosed as having cerebral atrophy. The ability to more accurately differentiate NPH from cerebral atrophy and normal aging, in a non-invasive manner, can potentially improve patient management by increasing selection accuracy of patients who can benefit from ventricular shunting.

A large number of invasive and non-invasive radiologic and radioisotope tests have been proposed to help identify subjects likely to respond to shunt. (See, e.g., References 33 and 34). Nevertheless, the high volume spinal tap still prevails more than 45 years later at many centers to help diagnose shunt responsive NPH, and to differentiate it from other clinical entities. However, this procedure can be invasive, and though very accurate when positive, has relatively low sensitivity (e.g., about 26-62%) for predicting a favorable surgical outcome, and therefore, may not be an optimal test for exclusion from shunt surgery. (See, e.g., References 35 and 36).

Several studies have looked at imaging markers that differentiate NPH from AD. (See, e.g., Reference 37). The MRI measure of increased CSF velocity traversing the aqueduct can be a diagnostic marker of hydrocephalus, and can also indicate that stroke volume measures correlate with shunt response. (See, e.g., Reference 33). Also the degree of dilatation of parahipoccampal fissures can be sensitive and specific for differentiating AD from NPH by both subjective and objective means. (See, e.g., Reference 38). More recently, diffusion tensor imaging has shown increased fractional anisotropy in several white matter (“WM”) tracts of NPH patients compared to normal controls. (See, e.g., References 39-42). However, there still remains a need for a simple and accurate method for diagnosing NPH.

Thus, it may be beneficial to provide an exemplary system, method and computer-accessible medium that can easily and accurately diagnose NPH, and which can overcome at least some of the deficiencies described herein above.

### SUMMARY OF EXEMPLARY EMBODIMENTS

Exemplary systems, methods, and computer-accessible mediums can be provided for determining a probability or a presence of a disease(s), which can include, for example, receiving information related to an image(s) of a brain of a patient(s), and determining the probability or the presence of the disease(s) in the patient(s) based on ventricular volume and gray matter of the brain. The disease can be normal pressure hydrocephalus or Alzheimer disease. The determining procedure can be based on the probability, which can be based on a prediction model(s). The prediction model can be a multinomial regression model.

In some exemplary embodiments of the present disclosure, the prediction model can be a linear regression model, which can include a binary linear regression model. A plurality of parameters of the prediction model(s) can be determined based on a maximum likelihood procedure, which can be an iterative maximum likelihood procedure. The parameters can include (i) the gray matter volume, (ii) the ventricular volume, (iii) a white matter volume, (iv) an age of a person associated with the disease(s) and (v) a gender of the person. The gray matter volume can be an absolute gray matter volume, the ventricular volume can be absolute ventricular volume and the white matter volume can be an absolute white matter volume. In certain exemplary embodiments of the present disclosure, the gray matter volume can be a relative gray matter volume, the ventricular volume can be a relative ventricular volume and the white matter volume can be a relative white matter volume.

In some exemplary embodiments of the present disclosure, the at least one prediction model includes a first prediction model and a second prediction model, and the first prediction model can be used to predict the probability or the presence of a first disease or a second disease and the second prediction model can be used to confirm results produced by the first prediction model. A second probability of an absence of the first disease or the second disease can be determined.

The image can be a magnetic resonance image of the brain of the patient(s). In some exemplary embodiments of the present disclosure, the ventricular volume can be determined by determining second information related to a segmentation of the information, determining third information based on a morphological closure procedure of the information, and determining a three-dimensional difference set between the third information and the second information.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIGS. 1A-1F are exemplary images illustrating how ventricular volume can be determine according to an exemplary embodiment of the present disclosure;

FIG. 2 is an exemplary graph illustrating an exemplary logistic curve according to an exemplary embodiment of the present disclosure;

FIG. 3 is an exemplary flow chart illustrating a method for predicting a disease according to an exemplary embodiment of the present disclosure;

FIG. 4 is an exemplary chart illustrating a comparison of segmentation and morphometric performance for a normal patient, normal pressure hydrocephalus, and Alzheimer's disease according to an exemplary embodiment of the present disclosure;

FIG. 5 is a exemplary chart illustrating the Evan's Index for a normal patient, normal pressure hydrocephalus, and Alzheimer's disease according to an exemplary embodiment of the present disclosure;

FIGS. 6A-6D are exemplary graphs illustrating Box-and-Whisker plots of volumes for different brain tissues according to an exemplary embodiment of the present disclosure;

FIG. 7 is a set of weighted and segmentation mask images for normal pressure hydrocephalus, Alzheimer disease, and healthy controls, according to an exemplary embodiment of the present disclosure;

FIG. 8 is a an exemplary scatter plot illustrating the distribution of absolute gray matter and ventricular volumes in normal pressure hydrocephalus, Alzheimer disease, and healthy controls, according to an exemplary embodiment of the present disclosure;

FIG. 9 is an exemplary graph illustrating receiver operating characteristic curves for the diagnostic discrimination of Alzheimer disease from normal pressure hydrocephalus patients according to an exemplary embodiment of the present disclosure;

FIG. 10 is an exemplary graph illustrating sulcal cerebral spinal fluid according to an exemplary embodiment of the present disclosure;

FIG. 11 is an image of a brain partitioned using sulcal masks according to an exemplary embodiment of the present disclosure;

FIG. 12 is diagram illustrating sulcal patterns in a brain according to an exemplary embodiment of the present disclosure;

FIG. 13 is a flow diagram of an exemplary method for determining a probability or a presence of a disease according to an exemplary embodiment of the present disclosure; and

FIG. 14 is an illustration of an exemplary block diagram of an exemplary system in accordance with certain exemplary embodiments of the present disclosure.

Throughout the drawings, the same reference numerals and characters, unless otherwise stated, are used to denote like features, elements, components or portions of the illustrated embodiments. Moreover, while the present disclosure will now be described in detail with reference to the Figures, it is done so in connection with the illustrative embodiments and is not limited by the particular embodiments illustrated in the Figures, when taken in conjunction with the appended claims.

### DETAILED DESCRIPTION OF EXEMPLARY EMBODIMENTS

### Exemplary NPH Group

For example, 91 consecutive patients were reviewed for symptoms of gait impairment, irrespective of the presence of cognitive or urologic dysfunction, and enlarged ventricles, and who underwent ventricular shunt surgery between January 2003 to December 2007. The diagnosis of probable NPH was made on the basis of enlarged ventricles, a characteristic of dyspraxic disorder and exclusion of other confounding diagnoses such as Parkinson's disease, cerebellar dysfunction, cerebrovascular disease, myelopathy or other metabolic diseases known to cause gait problems. Of the 91 patients, 15 met the following inclusion criteria: (i) gait improvement following ventricular shunt surgery, confirming the diagnosis of NPH, and (ii) availability of pre-surgical high-resolution (e.g., 1-mm slice) isotropic 3D T1-weighted magnetization-prepared rapid gradient echo (“MPRAGE”) MRI (e.g., Siemens Healthcare, Erlangen, Germany), needed for tissue segmentation. These 15 subjects formed the NPH group.

Gait impairment can be the principal symptom affecting older adults with NPH (see, e.g., Reference 16), and can be the clinical parameter most likely to improve with surgery. (See, e.g., References 17 and 18). For this reason, gait improvement was chosen as an outcome criterion for verifying, post hoc, the diagnosis of NPH. Thus, a positive response to shunt was defined as improvement in three different gait measures: (i) Functional Ambulation Performance (“FAP”) score, (ii) time to walk 30 feet and return, and (iii) ambulatory index. The FAP score is a quantitative, well validated composite gait measure based on step length, base, symmetry, velocity as well as other parameters. The FAP score can range from about 95-100 points in the healthy adult population (see, e.g., Reference 19), and was determined using the GaitRite System (e.g., CIR Systems, Inc., Havertown, Pa.) (20). The ambulatory index is an ordinal, symptom-based rating scale for characterizing ambulatory capacity. (See, e.g., Reference 21). The scale can range from 0 (e.g., normal gait) to 9 (e.g., inability to walk alone).

Cognitive status and urinary incontinence were used for clinical characterization. Cognitive function was evaluated using the Mini Mental State Examination (“MMSE”) and the Global Deterioration Scale (“GDS”). MMSE is a widely used 30-point scale for assessing mental capabilities, with healthy elderly adults scoring about 29-30 and mildly impaired subjects typically scoring about 25-28. GDS is a psychiatrist/neurologist-administered 7-point staging of the severity of cognitive impairment: 1-2=normal aging, 3=mild cognitive impairment and 4-7=progressive stages of dementia. (See, e.g., Reference 22). The average GDS for the NPH group was 3±0.2 (e.g., range of about 3-5).

Urinary incontinence was queried using a questionnaire administered at the time of initial clinical assessment and scored with a scale which ranges from 0 (e.g., no incontinence) to 9 (e.g., three or more incontinence episodes a day).

### Exemplary AD Group

AD subjects were selected and diagnosed based on structured clinical interviews, consistent with the NINCDS-ADRDA workgroup recommendations, and required symptoms of progressive impairments in multiple areas of cognition and difficulties with activities of daily living. All AD patients were rated as GDS>4, and ranged from about GDS 4 to 7 (e.g., average 4.5±0.2). Three rejection criteria that were applied were: (i) gait impairment, urinary incontinence or signs of Parkinsonism, (ii) major depression or other psychiatric diagnosis likely to confound cognitive assessment, and (iii) medical illnesses associated with CNS dysfunction, metabolic abnormalities, infarcts, severe leukoaraiosis or other structural brain changes likely to cause cognitive impairment. A total of 17 subjects were selected.

### Exemplary Healthy Controls

Healthy controls (“HC”) (n=18) were selected on the basis of a GDS score of about 1 or 2, and a normal neurologic examination. The selection of AD and HC subjects was blind to MRI findings. AD and HC subjects were selected from a larger available pool to maintain approximate age and gender match with the shunt-responsive NPH group.

### Exemplary MR Imaging Data Acquisition

All subjects had whole brain T1-weighted MRI acquired using MPRAGE sequence on Siemens 1.5 T and 3 T units (e.g., Avanto and TrioTim, Siemens AG, Erlangen, Germany). Images from NPH patients were selected from MRI studies obtained on average of about 1 to 5 months prior to shunt placement. MPRAGE images were acquired with the following parameters (e.g., at 1.5 T/3 T): TR 2100/2200 ms, TI 1100/1100 ms, TE 4/2.3 ms, matrix 256×179/256×256, FOV 256×256 mm2, 1 mm slice thickness, 200/260 Hz/pixel bandwidth, in 4:44/3:53 min total acquisition time.

### Exemplary MR Image Evaluation

Volumetric analyses of global GM, WM and CSF were performed using Statistical Parametric Mapping 8 (e.g., SPM8). (See, e.g., Reference 23). SPM tissue segmentation combines image contrast information with prior anatomical knowledge derived from a template image. The template consisted of MRIs of 152 subjects spatially registered and averaged in a common coordinate system that can approximate the Tailarach space (e.g., International Consortium for Brain Mapping 152, Montreal Neurological Institute).

In order to diagnose, or provide a probability of the occurrence of NPH, a scan or image of a brain (e.g., using an MRI) can be taken. Ventricular volume (“VNT”) can then be generated, for example, FireVoxel (see, e.g., Reference 24) using, e.g., three exemplary procedures: (i) an exemplary segmentation procedure (e.g., Bridge Burner procedure) can be used to segment the whole brain (see, e.g., Reference 24), (ii) an exemplary morphologic closure of the brain mask can be performed to include the ventricular spaces, (iii) a three-dimensional (“3D”) set difference between (b) and (a) can be taken as VNT. Discrimination algorithms can be applied to the VNT along with the GM of the patient, and a probability of NPH can then be generated. The probability can be based on a prediction model (e.g., a multinomial regression model), which can determine the probability that a patient has NPH, AD, or that the patient is normal.

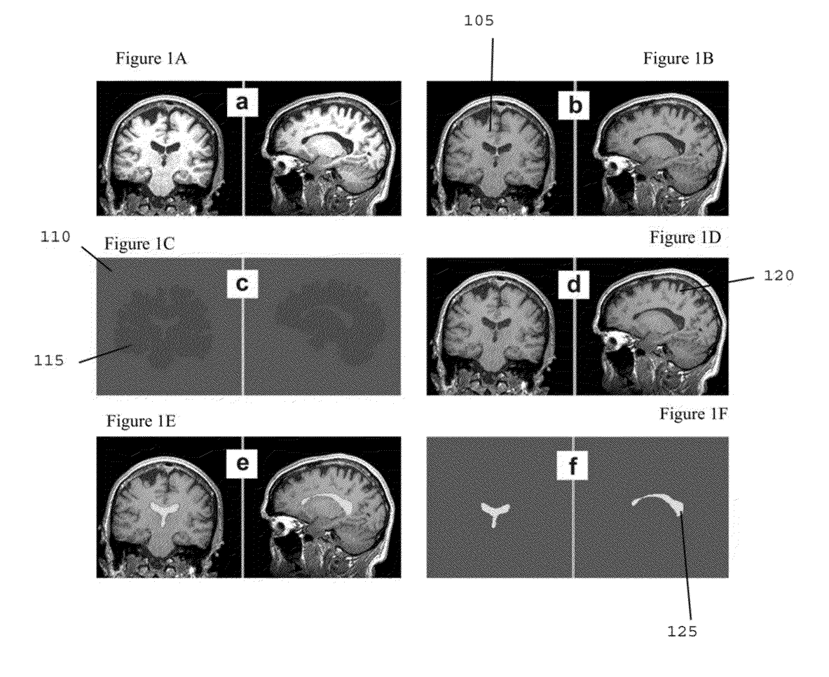

Total intracranial volume T can be defined as the sum of global GM, WM and CSF volumes, and can be used to generate the relative measures GM/T, WM/T, VNT/T. Computation of the ventricle mask and its volume can be a multi-step process, which is illustrated in FIGS. 1A-1F. As operations can be performed in a three dimensional space, for each procedure the FIGS. 1A-1F show two orthogonal views of the brain volume a coronal view on the left and a sagittal view on the right. The original T1-weighted MRIs is shown in FIG. 1A. The first procedure can include whole brain segmentation. This can result in a brain mask, shown FIGS. 1B and 1C. In FIG. 1B, the brain mask (element 105) is superimposed on the original MRI volumes. FIG. 1C illustrates the isolation of the binary brain mask, which can be represented in the computer as 0 (element 110) for background and 1 (element 115) for foreground. The brain mask can then be subjected to an exemplary “hole filling” operator. FIG. 1D illustrates the result of “hole filling” (element 120) superimposed on the MRIs. The result can be another, larger, binary mask.

The set difference operator can then be applied to the filled mask (e.g., FIG. 1D) and original brain mask (e.g., FIG. 1C). Each voxel that is marked as 1 in FIG. 1D but as 0 in FIG. 1B can be defined as the “ventricle mask”. This mask is shown in FIG. 1F by element 125, and it is superimposed in the MRIs in FIG. 1E. The VNT can be the number of voxels in the “ventricle mask” multiplied by the voxel volume.

Three radiologists, with 3-18 years of experience in the interpretation of brain MR images independently evaluated the MR images of all exemplary study subjects. Radiologists were asked to give one of the three diagnoses: hydrocephalus, atrophy or healthy elderly. Each radiologist was blind to the existing clinical diagnoses, and to the evaluation of the other two radiologists. Radiologist accuracy was computed as the percentage of correct responses.

### Exemplary Statistical Analyses

A multinomial regression model, and/or a logistic regression model can be used to predict probabilities of three different diagnoses: HC, NPH or AD, given brain measures (e.g., GM, WM, CSF and VNT). Multinomial regression can be similar to logistic regression, but it can be more general because the dependent variable may not be restricted to two categories. Model parameters can be estimated through an iterative maximum-likelihood procedure. (See, e.g., Reference 25). Logistic regression can be used to predict the presence of AD or NPH. The multinomial regression model can be used to confirm AD or NPH, and/or to predict of the person is HC.

### Exemplary Multinomial Regression Model

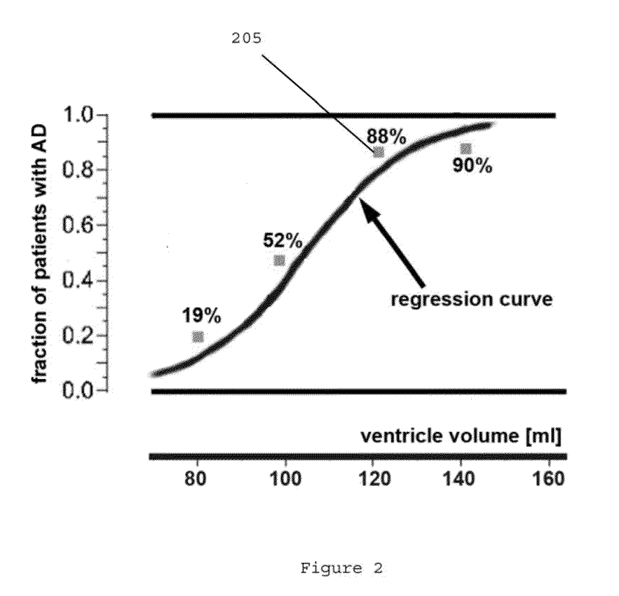

Measured ventricular volume, plotted on the horizontal x-axis of FIG. 2, can be used to predict AD. The relationship between the ventricle volume and the probability of AD can be described using a sigmoid-shape curve called a logistic curve. The logistic curve can be given by the equation:

$$P = \frac{1}{1 + {e}^{- ( a = bX )}} .$$

The curve can have two free parameters (e.g., a and b). The variable X can be the independent measure (e.g., ventricle volume). The probability P can range from 0 (e.g., when X approaches minus infinity) to 1 (e.g., when X approaches infinity). The parameter b can adjust how quickly the probability can change when changing X a single unit. The relation between X and P can be nonlinear.

The logistic curve can be generalized to several independent predictor measures (e.g., gray matter volume, white matter volume, ventricle volume), by expanding the exponent to produce

$$P = \frac{1}{1 + {e}^{- ( a + {b}_{1} {X}_{1} + {b}_{2} {X}_{2} + …}} .$$

Test data (e.g., shown by element 205 in FIG. 2), can be used to estimate the parameters a and b of the exemplary model above. In the example above, four categories of VNT can be shown (e.g., 80, 100, 120, 140 ml). 19% of patients with ventricle volume approximately 80 ml can be found to have AD; the proportion can increase to 52% for ventricle volume approximately 100 ml.

Unlike for linear regression, there can be no closed, direct, mathematical solution that can produce estimates of the parameters for the logistic or multinomial regressions. Instead, exemplary numerical analysis procedures can be used. These can function by using successive approximations to compute model parameters. For example, some initial estimates of the parameters can be chosen. Then, how close the data points are separated from the curve build can be computed from these initial parameters. These parameters can be shifted slightly in one direction (e.g., increasing or decreasing from initial estimates), and can be used to recalculate the fit of the data. If the fit improves, the same direction can be used, otherwise, the direction can be reversed. This exemplary process can be continued until the fit does not change much. Usually a change of 0.001 or 0.1% is small enough to tell the computer to stop. Sometimes the program is terminated after a certain number of iterations (e.g., about 100 iterations).

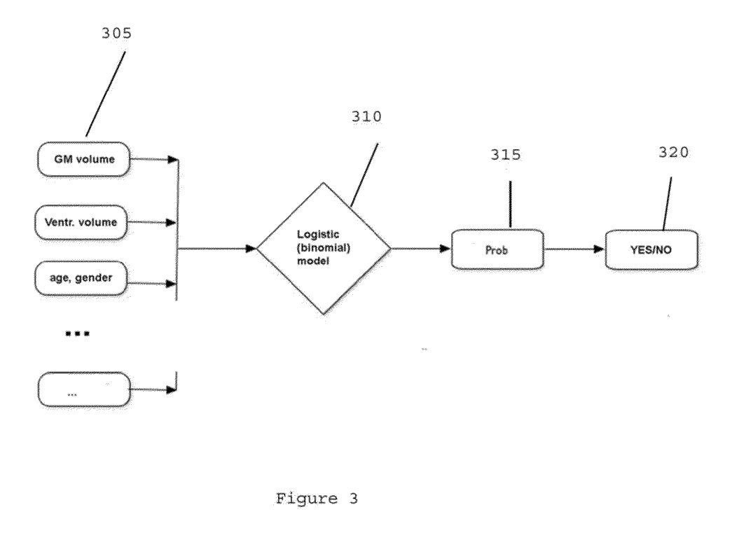

Once model parameters can be computed, the exemplary model can be used to predict the outcome for a new patient. For example, FIG. 3 illustrates a method for predicting the probability of a disease. At procedure 305 the predictor variable X can be measured (e.g., generally X1, X2, . . . ). At procedure 310, the variables can be entered into the exemplary equation above. At procedure 315 the probability can be computed, and at procedure 320, a determination of the presence or absence of a disease can be made.

Age and gender can also be entered as independent variables due to their known effect on brain size and atrophy. Separate regression models can be constructed for absolute and relative volumes as diagnostic predictors. Further logistic regressions and receiver operating characteristic (“ROC”) analyses can be done for: (i) predicting disease (e.g., AD or NPH) vs. HC, and (ii) discriminating AD from NPH.

Intraclass correlation coefficient (“ICC”) can be computed to assess the agreement among the radiologists. An analysis of variance (“ANOVA”) can be used for comparing mean values of individual variables across study groups. Tukey's honestly significant difference (“HSD”) test, a multiple comparison procedure, can be used in conjunction with an ANOVA to identify means that can be significantly different from each other. Statistical analyses can be done using the IBM SPSS statistical package, version 20 (e.g., IMB. Armonk, New York, USA).

### Exemplary Method

For example, N=72 consisted of 37 srNPH patients, 20 AD patients and 15 elderly HC. Each individual had high-resolution T1-weighted MRI acquired using MPRAGE sequences on Siemens 1.5/3 T units. Three observers with 1-3 years neuroanatomy experience were blinded to clinical data. Presence or absence of DESH was assessed by dichotomous global impression and five-point visual ordinal scales were used to assess prominence of SF and grouped high convexity/medial CS. EI and Callosal Angle (“CA”) were retrospectively and independently measured by each observer. GM volume was computed using SPM8, and VNT were segmented using locally developed software. For subjective metrics inter-observer variability was assessed using ICC. Each metric was tested in terms of its ability alone to diagnose each patient as either srNPH, AD, or HC. The categorical, morphometric and segmentation metrics were also group tested in multivariable predictive models using 3 way nominal regression. Finally, each model was tested, including a comparison of each observer's categorical and morphometric measurements with the automated volumetric segmentation.

### Exemplary Results

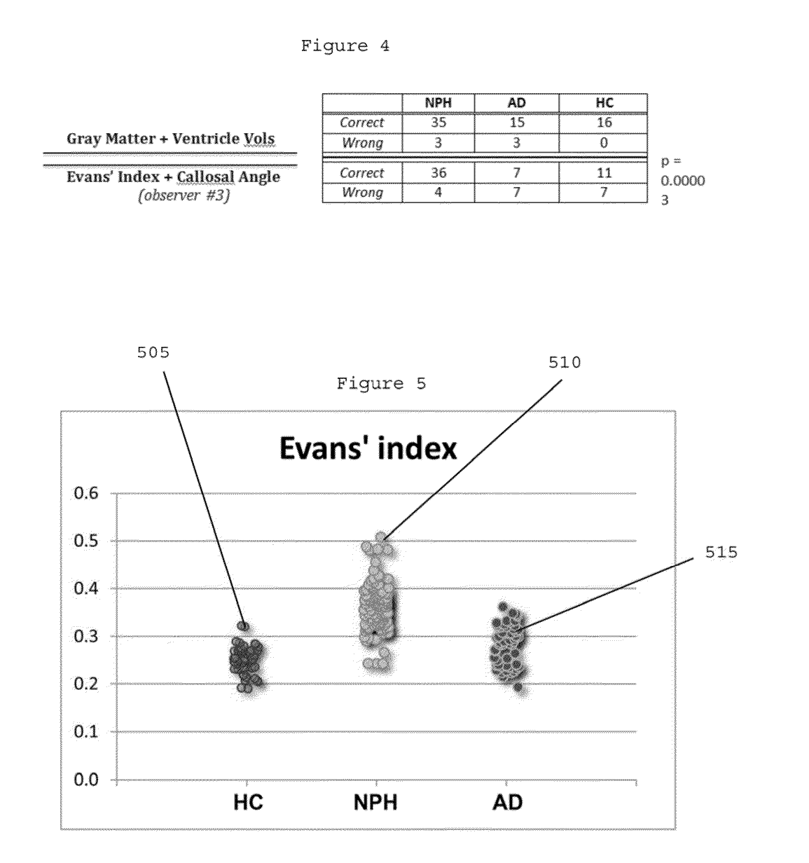

ICC showed very good interobserver agreement for DESH, sulcal prominence, EI and CA but only fair agreement for SF. Predictive accuracy (see e.g., Table 1) was very good for automated volumetric segmentation, with an overall accuracy of about 91.7%, and only fair in the other prediction models, with an overall accuracy of about 62.5-79.2%. Automated volumetric segmentation also showed superior accuracy in direct comparison with each observer's measurements, with a representative example for observer #3 in FIG. 4. There was a significant overlap of Evan's index (see, e.g., FIG. 5) across the three diagnostic groups (e.g., HC 505, NPH 510 and AD 515).

TABLE 1
Diagnostic Accuracy of Prediction Models

| | Overall | NPH | AD | HC |
|---|---|---|---|---|
| Prediction | Accuracy | Accuracy | Accuracy | Accuracy |
| Model | (%) | (%) | (%) | (%) |
| DESH, Sylvian | 72.2-76.4 | 81.6 | 38.9-72.2 | 50-93.8 |
| Fissures & Sulci | | | | |
| Combined | | | | |
| Evan's Index & | 72.2-79.2 | 92.1-94.7 | 38.9-55.6 | 68.8 |
| Callosal Angle | | | | |
| Combined | | | | |
| Evan's Index | 62.5-70.8 | 89.5-92.1 | 22.2-38.9 | 43.8-68.8 |
| Callosal Angle | 62.5-69.4 | 86.8-89.5 | 23.2-50.0 | 18.8-68.8 |
| Volumetric | 91.7 | 92.1 | 83.3 | 100 |
| Segmentation | | | | |

### Exemplary Demographics

Demographic and cognitive features of the subject groups are listed in Table 2 below. There were no significant group differences in age (e.g., ANOVA, F=0.524, p=0.595) or gender (e.g., Chi-Square=1.041, p=0.377). Mean MMSE scores were different across subject groups (e.g., ANOVA, F=23.9, p<0.001); they were lower in patients with AD than NPH (e.g., Tukey HSD, p<0.005) and also lower in NPH patients relative to HC (e.g., p<0.05).

TABLE 2
Demographics of the three subject groups

| | NPH | AD | Control |
|---|---|---|---|
| No. of subjects | 15 | 17 | 18 |
| Age* | 72.6 ± 8 | 72.1 ± 11 | 69.7 ± 7 |
| Age range | 56-84 | 53-87 | 59-84 |
| Men/women | 9/6 | 10/7 | 7/11 |
| MMSE* | 24.8 ± 4.6 | 19.2 ± 6.1 | 29.5 ± 0.7 |

(*mean ± SD)

### Exemplary Post-Shunt Changes In NPH Patients

The distribution of clinical characteristics measured in NPH patients before and after shunting is shown in Table 3 below. All 15 patients improved their gait testing following surgery. Data on cognitive function and urinary incontinence following shunt were available for 12 and 9 patients, respectively. There was evidence for cognitive improvement in five out of 12 patients, and urinary incontinence improvement in 6 out of 9 patients. However, when the average cognitive and urinary incontinence scores were considered as a group, no significant difference was seen between the pre- and post-operative period.

TABLE 3
Clinical data of NPH patients before and after surgery

| | Pre-shunt | Post-shunt | P |
|---|---|---|---|
| FAP | 74.4 ± 3.7 | 85.61 ± 3 | 0.03 |
| Timed gait | 23.2 ± 2.4 | 17.7 ± 1.2 | 0.01 |
| Ambulation index | 3 ± 0.1 | 2.2 ± 0.4 | 0.02 |
| MMSE | 24.8 ± 4.6 | 24.8 ± 1.8 | NS |
| GDS | 3 ± 0.2 | 2.8 ± 0.4 | NS |
| Urinary incont | 3.8 ± 0.6 | 2.1 ± 0.8 | NS |

(FAP = Functional Ambulation Performance score; Data are mean ± SD)

### Exemplary Brain Segmentation

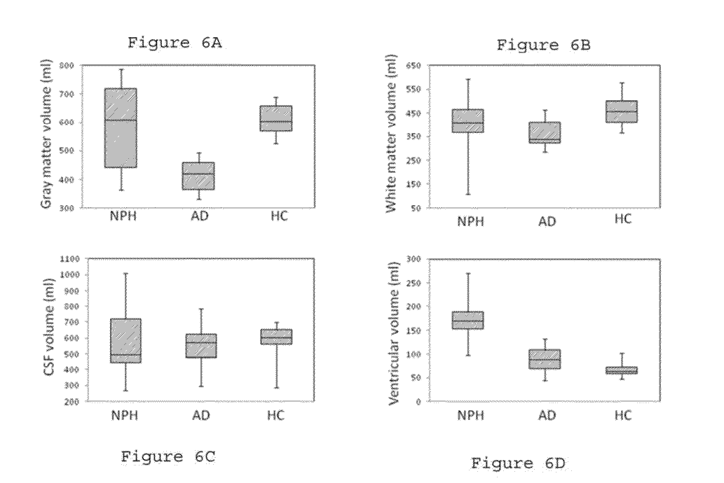

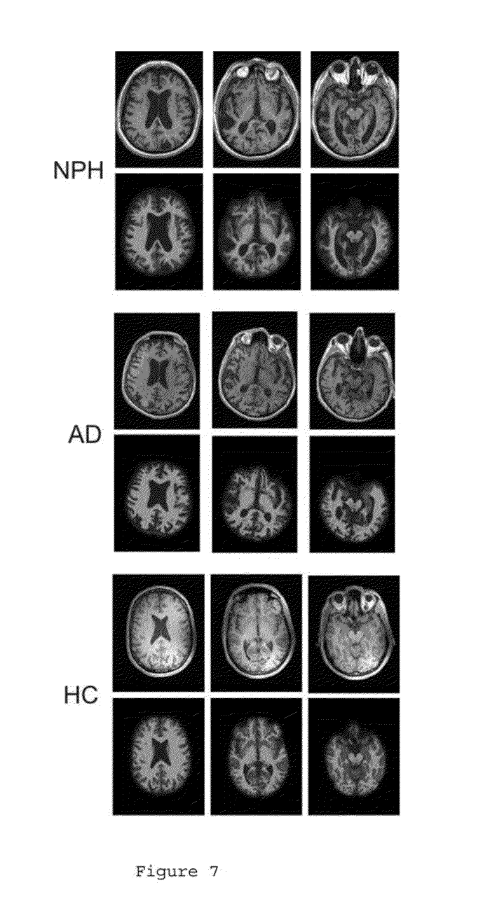

Tissue volumes for the three groups are shown in Table 4 below, as well as in the exemplary graphs of FIGS. 6A-6D. Representative transverse T1-weighted and segmentation images for each group are shown in FIG. 7. There was significant GM volume differences across the groups (e.g., ANOVA, F=23.2, p<0.001). The average GM volume in AD patients (e.g., 414 ml) was significantly lower than both NPH patients (e.g., 583 ml) and HC (e.g., 609 ml), with no significant difference between NPH patients and HC using a Tukey HSD follow-up test. One-factor ANOVA also showed overall group differences in WM (e.g., F=7.3, p<0.005), with AD average being lower than for HC, but no other significant post-hoc differences.

TABLE 4
Mean and SEM of the different tissue types

| Tissue type | NPH | AD | Control |
|---|---|---|---|
| GM | 583 ± 38 | 414 ± 12 | 609 ± 12 |
| WM | 408 ± 29 | 357 ± 14 | 460 ± 14 |
| CSF | 569 ± 54 | 565 ± 30 | 589 ± 23 |
| VNT | 177 ± 13 | 91 ± 7 | 68 ± 4 |
| TICV | 1560 ± 80 | 1336 ± 43 | 1657 ± 35 |

(GM = Gray matter; WM = White matter; CSF Cerebrospinal fluid; VNT = Ventricular volume; TICV = total intracranial volume)

There was no significant difference in total CSF across the groups (e.g., ANOVA, F=0.126, p=0.88). Ventricular volumes, on the other hand, were distributed unequally across the three groups (e.g., ANOVA, F=47.6, p<0.005). A Tukey post hoc test showed mean VNT in NPH patients (e.g., about 177 ml) to be significantly (e.g., p<0.001) larger than in HC (e.g., about 68 ml) and AD patients (e.g., about 91 ml), with a trend for greater VNT in AD compared to HC (e.g., p=0.11). Within each diagnostic group, tissue volumes were larger for male subjects than for female subjects.

### Exemplary Discrimination of Patient Groups Using Multivariate Analysis

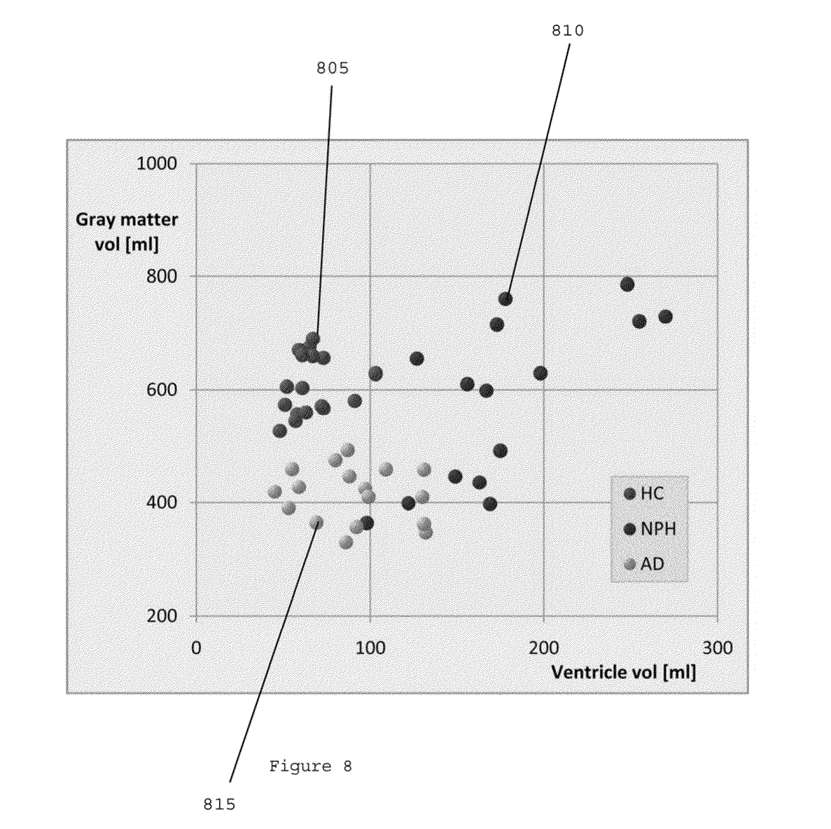

Exemplary results were plotted to examine patterns that might best distinguish NPH from AD and HC. A combination of VNT and GM volumes provided two-dimensional (“2D”) clustering for the three groups HC 805, NPH 810 and AD 815. (See e.g., FIG. 8). In the multinomial model, VNT and GM volumes, plus the categorical variable for gender, provided an excellent data fit (e.g., Chi-square 96.8, p<0.001, Cox and Snell R-square=0.856). Classification accuracy of the model is shown in Table 5 below. The model erred by misclassifying two out of fifteen NPH subjects as AD, yielding an overall accuracy of 96.3%. Entering WM or age did not improve results.

TABLE 5
Diagnostic accuracy (%) for the computer model based on brain
segmentation and for the three readers (qualitative assessment)

| | Overall | HC | NPH | AD |
|---|---|---|---|---|
| Model | 96.3 | 100.0 | 86.7 | 100.0 |
| Reader 1 | 78.0 | 72.2 | 80.0 | 82.4 |
| Reader 2 | 76.0 | 94.4 | 66.7 | 64.7 |
| Reader 3 | 68.0 | 94.4 | 73.3 | 35.3 |

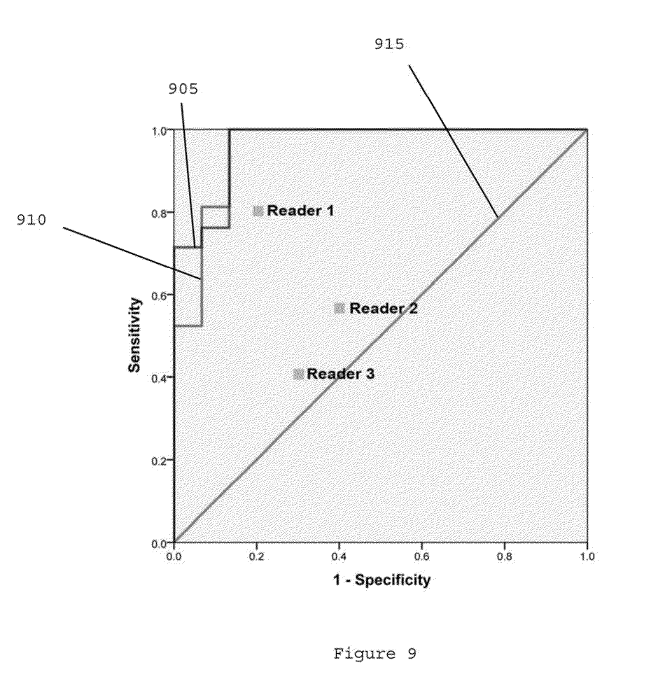

A separate binary logistic regression model was constructed to best discriminate NPH from AD patients using the same (e.g., GM, VNT, and gender) independent variables. FIG. 9 shows an exemplary graph of the ROC analysis for that binary prediction. The area under the ROC curve is, for example, 0.965. Exemplary ROC curve 905 can correspond to the binary logistic regression model that can include GM volume, ventricle volume and gender as predictors. ROC curve 910 omits subject's gender from the model. The exemplary plot illustrates the sensitivities and specificities of three radiologists (e.g., reader 1-3). Each radiologist performed less accurately than the nonlinear regression model. The diagonal line 915 indicates the characteristic curve of a chance prediction.

Similar results, but with slightly lower discrimination accuracy, were achieved with relative measures, for example, after normalizing for cranial cavity volume.

### Exemplary Comparison of Qualitative Assessment With Regression Model Based on Segmentation Measures

When faced with the classification of subjects as NPH, AD and healthy elderly, the overall diagnostic accuracy for the three radiologists was about 78%, about 76% and about 68%, respectively. (See e.g., Table 5). The overall accuracy of the three readers was about 74%, which was significantly lower than the model (e.g., p<0.005). Interobserver variability was assessed using the ICC. The three readers had a “fair agreement” with an ICC of 0.51 (e.g., about 95% confidence interval between 0.34 and 0.66). There were discordant readings in 10 out of 50 cases (e.g., 20%) between readers 1 and 2, in 16 (e.g., 32%) cases between readers 1 and 3, and 14 (e.g., 28%) cases between readers 2 and 3.

### Exemplary Discrimination Using Sulcal Patterns

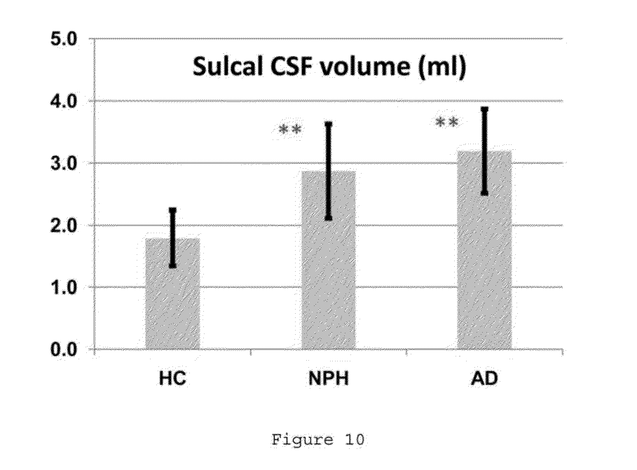

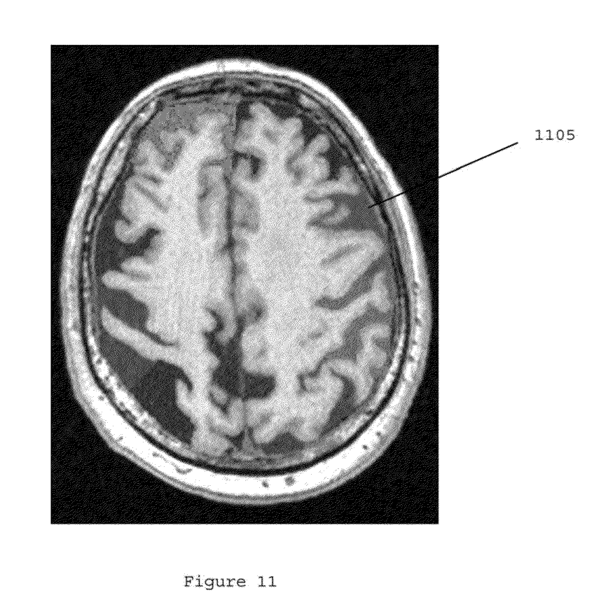

As shown in FIG. 10, sulcal (e.g., extra-ventricular) CSF can also be considered. Sulcal volume can be significantly larger in AD compared to healthy controls (e.g., p=0.000001, T=6.02). Sulcal volume can also be abnormal in NPH (e.g., p=0.0002, T=4.17), but there can be no significant volume difference between NPH and AD.

FIG. 11 is an image of a brain partitioned using sulcal masks. Following radiologic observations, sulcal pattern can be considered that are initially obtained by partitioning sulcal masks into wedges. Sulcal CSF can yield a series D(θ) (e.g., element 1105), where V can be the sulcal CSF volume and θ the polar angle. The distribution D(θ) can be subjected to pattern analysis.

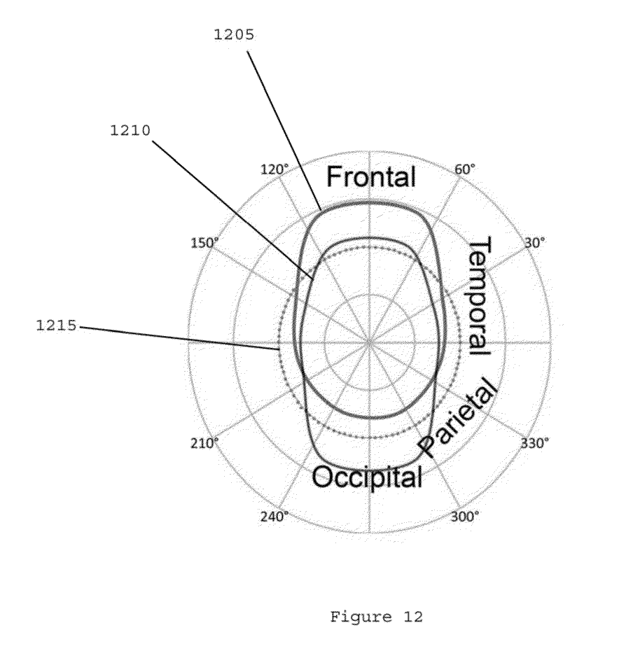

FIG. 12 is diagram illustrating sulcal patterns in a brain. Element 1205 can represent NPH patients. Element 1210 can represent probable AD patients. Element 1215 can represent a homogenous distribution. As shown in FIG. 12, NPH can show relatively larger sulci in the frontal lobes (element 705). Overall the pattern can be more circular (homogenous element 715). AD brains can illustrate relatively larger sulci in parietal and occipital lobes (element 710).

### Exemplary Discussion

Traditional imaging evaluation of NPH has relied on visual assessment of ventricular size and other subjective parameters, which alone may not be sufficient to diagnose NPH. (See, e.g., Reference 26). Despite expanding imaging literature, there can be wide variation in radiologists' perception of expected imaging features of normal aging, brain atrophy and NPH, with considerable interreader variability in accuracy as demonstrated above, in which there were discordant readings in 20 to 32% of cases. This can shed light on the underpinnings of this large variability in the interpretation of the scans of elderly patients. Specifically, these three groups varied in the distribution of CSF between the ventricles and subarachnoid spaces, and in the visually undetectable differences in GM volume. The exemplary system, method and computer-accessible medium, according to an exemplary embodiment of the present disclosure, can demonstrate that shunt-responsive NPH patients can have markedly larger ventricles than AD and HC. Additionally, GM volume in NPH can be higher than in AD, and similar to HC. The exemplary multivariate regression model based on a combination of GM and ventricular volumes had significantly higher accuracy in differentiating AD, NPH and HC when compared to the qualitative assessment by the radiologists.

Global and regional GM and WM atrophy in AD, and the changes in their volume, can correlate with clinical progression of the disease. (See, e.g., References 14 and 27-29). However, the evidence for changes of segmented brain volumes in NPH is not well documented. Using voxel-based morphometry, enlarged ventricles and sylvian fissures, stenotic high convexity sulci, and regionally variable GM density in probable NPH patients can be found relative to probable AD. (See, e.g., Reference 30). It was previously determined that a combination of cortical thickness and ventricular volume distinguished five NPH patients from five AD and five Parkinson disease patients. (See, e.g., Reference 31). Also, there can be greater cortical thinning in AD when compared to NPH patients with positive response to CSF tap. (See, e.g., Reference 32). The exemplary results can be in overall agreement with previous studies in that GM volume can be reduced in AD compared to NPH.

The exemplary finding can be beneficial for radiologic assessment for two exemplary reasons. First, for example, T1-weighted images were used that are acquired routinely in clinical practice. Second, the exemplary segmentation procedure can be quick, relatively simple to apply, and readily available. Consequently, automated global analysis can be applied in the routine clinical setting to enhance diagnostic accuracy. The exemplary system, method, and computer-accessible medium, can thus improve the selection of those patients who would benefit from shunt implantation.

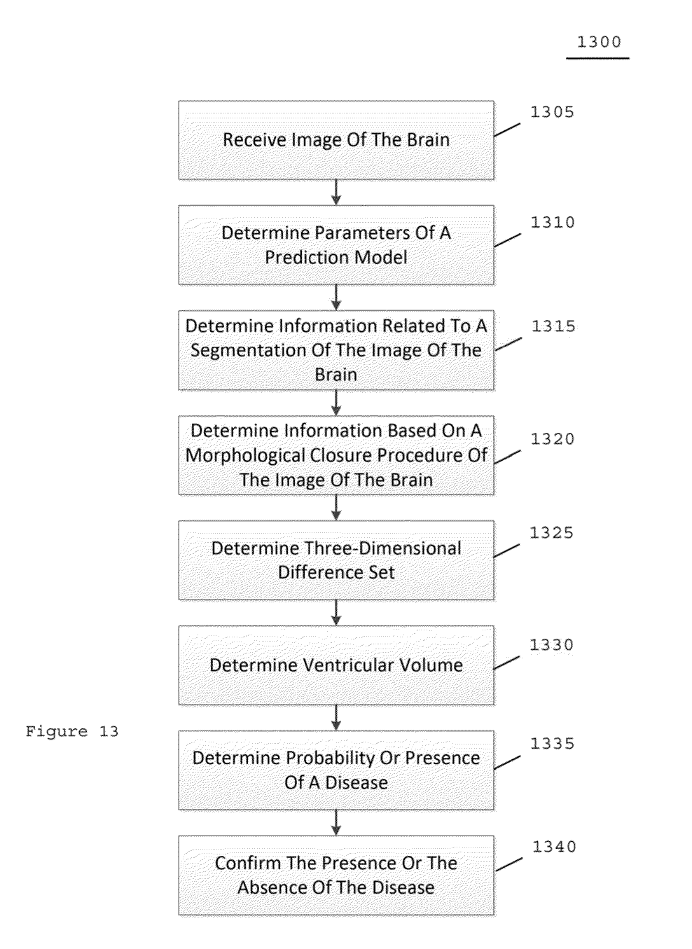

FIG. 13 is a flow diagram of an exemplary method 1300 for determining a probability or a presence a disease according to an exemplary embodiment of the present disclosure. For example, at procedure 1305 an image of the brain can be received from, for example a magnetic resonance imaging apparatus. Model parameters used to predict the probability or the presence can be determines at procedure 1310. Information related to a segmentation of the image of the brain can be determined at procedure 1315, and information based on a morphological closure procedure of the image of the brain can be determined at procedure 1320. At procedure 1325, a 3D difference set between the information determined at procedures 1315 and 1320 can be determined, which can be used to determine a ventricular volume at procedure 1330. At procedure 1335, a probability or presence of the disease can be determined, which can be confirmed at procedure 1340.

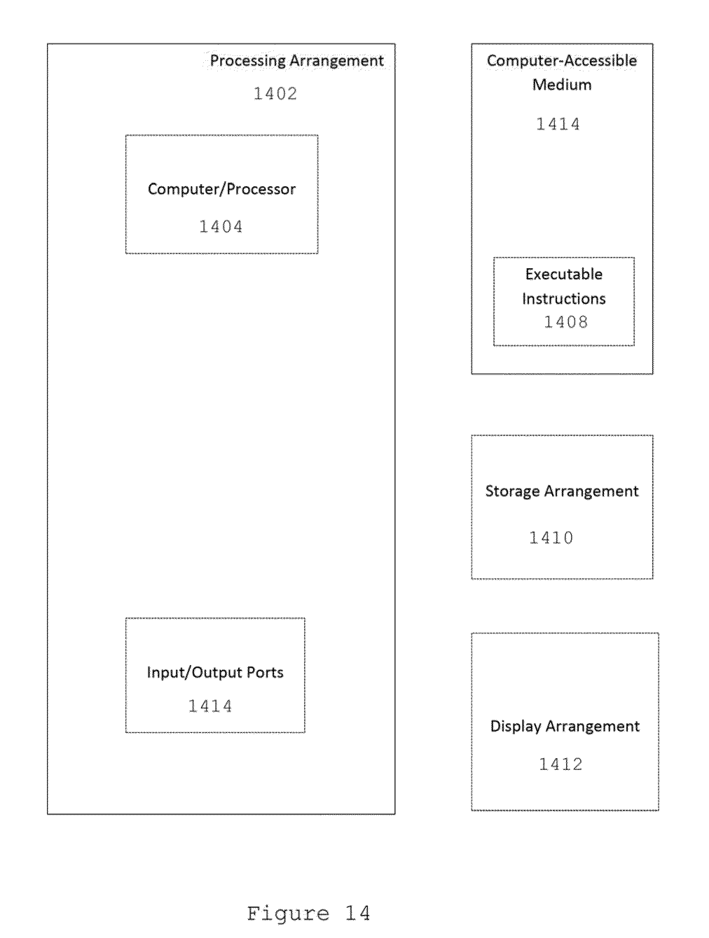

FIG. 14 shows a block diagram of an exemplary embodiment of a system according to the present disclosure. For example, exemplary procedures in accordance with the present disclosure described herein can be performed by a processing arrangement and/or a computing arrangement 1402. Such processing/computing arrangement 1402 can be, for example entirely or a part of, or include, but not limited to, a computer/processor 1404 that can include, for example one or more microprocessors, and use instructions stored on a computer-accessible medium (e.g., RAM, ROM, hard drive, or other storage device).

As shown in FIG. 14, for example a computer-accessible medium 1406 (e.g., as described herein above, a storage device such as a hard disk, floppy disk, memory stick, CD-ROM, RAM, ROM, etc., or a collection thereof) can be provided (e.g., in communication with the processing arrangement 1402). The computer-accessible medium 1406 can contain executable instructions 1408 thereon. In addition or alternatively, a storage arrangement 1410 can be provided separately from the computer-accessible medium 1406, which can provide the instructions to the processing arrangement 1402 so as to configure the processing arrangement to execute certain exemplary procedures, processes and methods, as described herein above, for example.

Further, the exemplary processing arrangement 1402 can be provided with or include an input/output arrangement 1414, which can include, for example a wired network, a wireless network, the internet, an intranet, a data collection probe, a sensor, etc. As shown in FIG. 14, the exemplary processing arrangement 1402 can be in communication with an exemplary display arrangement 1412, which, according to certain exemplary embodiments of the present disclosure, can be a touch-screen configured for inputting information to the processing arrangement in addition to outputting information from the processing arrangement, for example. Further, the exemplary display 1412 and/or a storage arrangement 1410 can be used to display and/or store data in a user-accessible format and/or user-readable format.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, e.g., data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### EXEMPLARY REFERENCES

1. Hakim S, Adams R D. The special clinical problem of symptomatic hydrocephalus with normal cerebrospinal fluid pressure. Observations on cerebrospinal fluid hydrodynamics. Journal of the neurological sciences. 1965; 2(4):307-27.
2. Adams R D, Fisher C M, Hakim S, Ojemann R G, Sweet W H. Symptomatic Occult Hydrocephalus with “Normal” Cerebrospinal-Fluid Pressure. A Treatable Syndrome. The New England journal of medicine. 1965; 273:117-26.
3. Finney G R. Normal pressure hydrocephalus. International review of neurobiology. 2009; 84:263-81.
4. Savolainen S, Hurskainen H, Paljarvi L, Alafuzoff I, Vapalahti M. Five-year outcome of normal pressure hydrocephalus with or without a shunt: predictive value of the clinical signs, neuropsychological evaluation and infusion test. Acta neurochirurgica. 2002; 144(6):515-23; discussion 23.
5. Aygok G, Marmarou A, Young H F. Three-year outcome of shunted idiopathic NPH patients. Acta neurochirurgica Supplement. 2005; 95:241-5.
6. Malm J, Kristensen B, Stegmayr B, Fagerlund M, Koskinen L O. Three-year survival and functional outcome of patients with idiopathic adult hydrocephalus syndrome. Neurology. 2000; 55(4):576-8.
7. Meier U, Miethke C. Predictors of outcome in patients with normal-pressure hydrocephalus. Journal of clinical neuroscience: official journal of the Neurosurgical Society of Australasia. 2003; 10(4):453-9.
8. Kahlon B, Sjunnesson J, Rehncrona S. Long-term outcome in patients with suspected normal pressure hydrocephalus. Neurosurgery. 2007; 60(2):327-32; discussion 32.
9. Tisell M, Hellstrom P, Ahl-Borjesson G, et al. Long-term outcome in 109 adult patients operated on for hydrocephalus. British journal of neurosurgery. 2006; 20(4):214-21.
10. Hebb A O, Cusimano M D. Idiopathic normal pressure hydrocephalus: a systematic review of diagnosis and outcome. Neurosurgery. 2001; 49(5):1166-84; discussion 84-6.
11. George A E, Holodny A, Golomb J, de Leon M J. The differential diagnosis of Alzheimer's disease. Cerebral atrophy versus normal pressure hydrocephalus. Neuroimaging clinics of North America. 1995; 5(1):19-31.
12. Holodny A I, George A E, de Leon M J, Golomb J, Kalnin A J, Cooper P R. Focal dilation and paradoxical collapse of cortical fissures and sulci in patients with normal-pressure hydrocephalus. Journal of neurosurgery. 1998; 89(5):742-7.
13. Toma A K, Holl E, Kitchen N D, Watkins L D. Evans' index revisited: the need for an alternative in normal pressure hydrocephalus. Neurosurgery. 2011; 68(4):939-44.
14. Rusinek H, de Leon M J, George A E, et al. Alzheimer disease: measuring loss of cerebral gray matter with MR imaging. Radiology. 1991; 178(1):109-14.
15. George A E, de Leon M J, Ferris S H, Kricheff, I I. Parenchymal CT correlates of senile dementia (Alzheimer disease): loss of gray-white matter discriminability. AJNR American journal of neuroradiology. 1981; 2(3):205-13.
16. Fisher C M. Hydrocephalus as a cause of disturbances of gait in the elderly. Neurology. 1982; 32(12):1358-63.
17. Graff-Radford N R, Godersky J C. Normal-pressure hydrocephalus. Onset of gait abnormality before dementia predicts good surgical outcome. Archives of neurology. 1986; 43(9):940-2.
18. Golomb J, Wisoff J, Miller D C, et al Alzheimer's disease comorbidity in normal pressure hydrocephalus: prevalence and shunt response. Journal of neurology, neurosurgery, and psychiatry. 2000; 68(6):778-81.
19. Nelson A J. Analysis of movement through utilisation of clinical instrumentation. Physiotherapy. 1976; 62(4):123-4.
20. Nelson A J, Zwick D, Brody S, et al. The validity of the GaitRite and the Functional Ambulation Performance scoring system in the analysis of Parkinson gait. NeuroRehabilitation. 2002; 17(3):255-62.
21. Hauser S L, Dawson D M, Lehrich J R, Beal M F, Kevy S V, Weiner H L. Immunosuppression and plasmapheresis in chronic progressive multiple sclerosis. Design of a clinical trial. Archives of neurology. 1983; 40(11):687-90.
22. Reisberg B, Ferris S H, de Leon M J, Crook T. The Global Deterioration Scale for assessment of primary degenerative dementia. The American journal of psychiatry. 1982; 139(9):1136-9.
23. Ashburner J, Friston K J. Unified segmentation. Neurolmage. 2005; 26(3):839-51.
24. Mikheev A, Nevsky G, Govindan S, Grossman R, Rusinek H. Fully automatic segmentation of the brain from T1-weighted MRI using Bridge Burner algorithm. Journal of magnetic resonance imaging: JMRI. 2008; 27(6):1235-41.
25. A. A. Categorical Data Analysis. 2nd ed. New York: John Wiley & Sons, 2002.
26. Tarnaris A, Kitchen N D, Watkins L D. Noninvasive biomarkers in normal pressure hydrocephalus: evidence for the role of neuroimaging. Journal of neurosurgery. 2009; 110(5):837-51.
27. Tanabe J L, Amend D, Schuff N, et al. Tissue segmentation of the brain in Alzheimer disease. AJNR American journal of neuroradiology. 1997; 18(1):115-23.
28. Karas G B, Burton E J, Rombouts S A, et al. A comprehensive study of gray matter loss in patients with Alzheimer's disease using optimized voxel-based morphometry. Neurolmage. 2003; 18(4):895-907.
29. Balthazar M L, Yasuda C L, Pereira F R, Pedro T, Damasceno B P, Cendes F. Differences in grey and white matter atrophy in amnestic mild cognitive impairment and mild Alzheimer's disease. European journal of neurology: the official journal of the European Federation of Neurological Societies. 2009; 16(4):468-74.
30. Ishii K, Kawaguchi T, Shimada K, et al. Voxel-based analysis of gray matter and CSF space in idiopathic normal pressure hydrocephalus. Dementia and geriatric cognitive disorders. 2008; 25(4):329-35.
31. Moore D W, Kovanlikaya I, Heier L A, et al. A pilot study of quantitative MRI measurements of ventricular volume and cortical atrophy for the differential diagnosis of normal pressure hydrocephalus. Neurology research international. 2012; 2012:718150.
32. Kang K, Yoon U, Lee J M, Lee H W. Idiopathic normal-pressure hydrocephalus, cortical thinning, and the cerebrospinal fluid tap test. Journal of the neurological sciences. 2013; 334(1-2):55-62.
33. Bradley W G, Jr., Scalzo D, Queralt J, Nitz W N, Atkinson D J, Wong P. Normal-pressure hydrocephalus: evaluation with cerebrospinal fluid flow measurements at MR imaging. Radiology. 1996; 198(2):523-9.
34. Bradley W G, Jr. Diagnostic tools in hydrocephalus. Neurosurgery clinics of North America. 2001; 12(4):661-84, viii.
35. Walchenbach R, Geiger E, Thomeer R T, Vanneste J A. The value of temporary external lumbar CSF drainage in predicting the outcome of shunting on normal pressure hydrocephalus. Journal of neurology, neurosurgery, and psychiatry. 2002; 72(4):503-6.
36. Marmarou A, Bergsneider M, Klinge P, Relkin N, Black P M. The value of supplemental prognostic tests for the preoperative assessment of idiopathic normal-pressure hydrocephalus. Neurosurgery. 2005; 57(3 Suppl):S17-28; discussion ii-v.
37. Bradley W G. Normal pressure hydrocephalus: new concepts on etiology and diagnosis. AJNR American journal of neuroradiology. 2000; 21(9):1586-90.
38. Holodny A I, Waxman R, George A E, Rusinek H, Kalnin A J, de Leon M. MR differential diagnosis of normal-pressure hydrocephalus and Alzheimer disease: significance of perihippocampal fissures. AJNR American journal of neuroradiology. 1998; 19(5):813-9.
39. Hattori T, Ito K, Aoki S, et al. White matter alteration in idiopathic normal pressure hydrocephalus: tract-based spatial statistics study. AJNR American journal of neuroradiology. 2012; 33(1):97-103.
40. Osuka S, Matsumura A, Ishikawa E, Matsushita A. Diffusion tensor imaging in patients with adult chronic idiopathic hydrocephalus. Neurosurgery. 2010; 67(5):E1474.
41. Hattingen E, Jurcoane A, Melber J, et al. Diffusion tensor imaging in patients with adult chronic idiopathic hydrocephalus. Neurosurgery. 2010; 66(5):917-24.
42. Kim M J, Seo S W, Lee K M, et al. Differential diagnosis of idiopathic normal pressure hydrocephalus from other dementias using diffusion tensor imaging. AJNR American journal of neuroradiology. 2011; 32(8):1496-503.

1. A non-transitory computer-accessible medium having stored thereon computer-executable instructions for determining at least one of a probability or a presence of at least one disease, wherein, when a computer hardware arrangement executes the instructions, the computer arrangement is configured to perform procedures comprising:
receiving information related to at least one image of a brain of at least one patient;
determining the at least one of the probability or the presence of the at least one disease in the at least one patient based on ventricular volume and gray matter volume of the brain.

2. The computer-accessible medium of claim 1, wherein the at least one disease is normal pressure hydrocephalus (“NPH”).

3. The computer-accessible medium of claim 1, wherein the at least one disease is Alzheimer disease.

4. The computer-accessible medium of claim 1, wherein the determining procedure is based on the probability.

5. The computer-accessible medium of claim 4, wherein the probability is based on at least one prediction model.

6. The computer-accessible medium of claim 5, wherein the prediction model is a multinomial regression model.

7. The computer-accessible medium of claim 5, wherein the prediction model is a linear regression model.

7. The computer-accessible medium of claim 7, wherein the linear regression model is a binary linear regression model.

9. The computer-accessible medium of claim 5, wherein the computer arrangement is further configured to determine a plurality of parameters of the at least one prediction model, using a maximum likelihood procedure.

10. The computer-accessible medium of claim 9, wherein the maximum likelihood procedure is an iterative maximum likelihood procedure.

11. The computer-accessible medium of claim 9, wherein the parameters include (i) the gray matter volume, (ii) the ventricular volume, (iii) a white matter volume, (iv) an age of a person associated with the at least one disease, and (v) a gender of the person.

12. The computer-accessible medium of claim 11, wherein the gray matter volume is an absolute gray matter volume, wherein the ventricular volume is an absolute ventricular volume, and wherein the white matter volume is an absolute white matter volume.

13. The computer-accessible medium of claim 11, wherein the gray matter volume is a relative gray matter volume, wherein the ventricular volume is a relative ventricular volume, and wherein the white matter volume is a relative white matter volume.

14. The computer-accessible medium of claim 5, wherein the at least one prediction model includes a first prediction model and a second prediction model, and wherein the computer arrangement is further configured to (i) utilize the first prediction model to predict the at least one of the probability or the presence of a first disease or a second disease, and (ii) utilize the second prediction model to confirm results produced by the first prediction model.

15. The computer-accessible medium of claim 14, wherein the computer arrangement is further configured to determine a second probability of an absence of at least one of the first disease or the second disease.

16. The computer-accessible medium of claim 1, wherein the image is a magnetic resonance image of the brain of the at least one patient.

17. The computer-accessible medium of claim 1, wherein the computer arrangement is further configured to determine the ventricular volume by:
determining second information related to a segmentation of the information;
determining third information based on a morphological closure procedure of the information; and
determining a three-dimensional difference set between the third information and the second information.

18. The computer-accessible medium of claim 1, wherein the computer arrangement is further configured to determine the at least one of the probability or the presence of the at least one disease in the at least one patient based on a sulcal cerebral spinal fluid volume.

19. A method for determining at least one of a probability or a presence of at least one disease, comprising:
receiving information related to at least one image of a brain of at least one patient;
using a computer hardware arrangement, determining the at least one of the probability or the presence of the at least one disease in the at least one patient based on ventricular volume and gray matter of the brain.

20. A system for determining at least one of a probability or a presence of at least one disease, comprising:
a computer hardware arrangement configured to:
receive information related to at least one image of a brain of at least one patient;
determine the at least one of the probability or the presence of the at least one disease in the at least one patient based on ventricular volume and gray matter of the brain.
